(19) United States
(12) Patent Application Publication
(10) Pub. No.: US 2023/0058381 A1
(43) Pub. Date: Feb. 23, 2023

(54) SYSTEMS AND METHODS FOR THE INJECTION OF VISCOUS FLUIDS

(71) Applicant: Massachusetts Institute of Technology, Cambridge, MA (US)

(72) Inventors: Kripa K. Varanasi, Lexington, MA (US); Vishnu Jayaprakash, Cambridge, MA (US); Maxime Costalonga, Cambridge (GB)

(73) Assignee: Massachusetts Institute of Technology, Cambridge, MA (US)

(21) Appl. No.: 17/796,199

(22) PCT Filed: Jan. 28, 2021
(86) PCT No.: PCT/US2021/015397

§ 371 Date: Jul. 28, 2022

Related U.S. Application Data
(60) Provisional application No. 62/967,239, filed on Jan. 29, 2020.

(51) Int. Cl. A61M 5/24; A61M 5/34

(52) U.S. Cl. CPC A61M 5/2448; A61M 5/34

(57) ABSTRACT

Disclosed herein are systems and methods for the injection of viscous fluids. For example, inventive systems and methods for injecting viscous fluids, such as concentrated drug formulations, via droplet lubrication are described.

### RELATED APPLICATIONS

This application claims priority under 35 U.S.C. § 119(e) to U.S. Provisional Patent Application No. 62/967,239, filed Jan. 29, 2020, which is hereby incorporated by reference in its entirety.

### TECHNICAL FIELD

Systems and methods for the injection of viscous fluids are generally described.

### SUMMARY

Disclosed herein are systems and methods for the injection of viscous fluids. For example, inventive systems and methods for injecting viscous fluids, such as concentrated drug formulations, via droplet lubrication are described. In some embodiments, injectability of an inner fluid (e.g., a concentrated drug formulation) is desired. In certain embodiments, the systems and methods comprise an outer fluid axially surrounding the inner fluid. In certain cases, the outer fluid lubricates the flow of the inner fluid by preferentially wetting, relative to the inner fluid, the interior surface of a needle and/or chamber through which the fluids are transported. In some cases, the inner fluid does not contact the interior surface of the needle and/or chamber through which the inner fluid is transported. The subject matter of the present invention involves, in some cases, interrelated products, alternative solutions to a particular problem, and/or a plurality of different uses of one or more systems and/or articles.

Certain embodiments are related to articles for delivery of a fluid. In some embodiments, the article for delivery of a fluid, comprises: a chamber; a needle fluidically connected to the chamber; an inner fluid extending from the chamber into the needle; and an outer fluid extending from the chamber into the needle and axially surrounding the inner fluid; wherein the outer fluid preferentially wets an interior surface of the needle relative to the inner fluid.

In some embodiments, the article for delivery of a fluid, comprises: a chamber; and a needle fluidically connected to the chamber; wherein the article is configured such that, when an inner fluid and an outer fluid are transported through the needle, the outer fluid axially surrounds the inner fluid, and the outer fluid preferentially wets an interior surface of the needle relative to the inner fluid.

In certain embodiments, the article for delivery of a fluid, comprises: a chamber; a needle fluidically connected to the chamber; an inner fluid extending from the chamber into the needle and flowing through the needle; and an outer fluid extending from the chamber into the needle, axially surrounding the inner fluid, and flowing through the needle; wherein the outer fluid mixes with the inner fluid at most 50% while in the needle.

In certain embodiments, the article for delivery of a fluid, comprises: a chamber; and a needle fluidically connected to the chamber; wherein the article is configured such that, when an inner fluid and an outer fluid are transported through the needle, the outer fluid axially surrounds the inner fluid, and the outer fluid mixes with the inner fluid at most 50% while in the needle.

In some embodiments, the article for delivery of a fluid, comprises: a chamber; a needle fluidically connected to the chamber; an inner fluid extending from the chamber into the needle and flowing through the needle; and an outer fluid extending from the chamber into the needle, axially surrounding the inner fluid, and flowing through the needle; wherein the article has an eccentricity parameter (E) of less than 1 when a longitudinal axis of the needle is within 45 degrees of a line perpendicular to gravity for at least one period of time.

In certain embodiments, the article for delivery of a fluid, comprises: a chamber; and a needle fluidically connected to the chamber; wherein an interior surface of the needle comprises a texture that imparts wettability for at least one fluid when a droplet of that fluid is present on the interior surface of the needle in another fluid.

In some embodiments, the article for delivery of a fluid, comprises: a chamber; and a needle fluidically connected to the chamber; wherein an interior surface of the needle comprises a coating that imparts wettability for at least one fluid when a droplet of that fluid is present on the interior surface of the needle in another fluid.

In some embodiments, the article for delivery of a fluid, comprises: a chamber; a needle fluidically connected to the chamber; an inner fluid comprising a liquid and a species suspended and/or dissolved in the liquid, the inner fluid extending from the chamber into the needle and flowing through the needle; and an outer fluid comprising the liquid and extending from the chamber into the needle, the outer fluid axially surrounding the inner fluid and flowing through the needle; wherein the outer fluid does not contain the species or contains the species at a molar concentration that is at least 50% lower than a molar concentration of the species within the inner fluid.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is, in accordance with some embodiments, a schematic illustration of an article for delivery of a fluid, comprising chamber 101, needle 102, inner fluid 103, and outer fluid 104.

FIG. 1B is, in accordance with some embodiments, a cross-sectional view of the needle, depicting the outer fluid and inner fluid within the needle.

FIG. 2A is a time-lapse image demonstrating the difficulty in the manual injection of a high viscosity solution (52 cP glycerol/water, top) compared to a low viscosity solution (1 cP water, bottom) through a 27 G needle. The time-lapse image was taken over 7 seconds of manual injection into an absorbent sponge. The operator applied the maximum pinching force possible (approximately 50N).

FIG. 2B is a plot of the manual injection force required to inject eleven high concentration monoclonal antibody solutions of the IgG1 isotype.

FIG. 2C is, in accordance with some embodiments, a schematic of unlubricated flow and axially lubricated flow through a needle.

FIG. 2D is, in accordance with some embodiments, a plot of pressure reduction coefficient (η) versus the ratio of the volumetric flow rate of the outer fluid to that of the inner fluid (Qₒ/Qᵢ) for various viscosity ratios (λ).

FIG. 3A is, in accordance with some embodiments, a schematic illustration of an article used to inject viscous fluid.

FIG. 3B plots, in accordance with some embodiments, the viscosity ratios (λ) versus the ratio of the volumetric flow rate of the outer fluid to that of the inner fluid (Qₒ/Qᵢ), and shows which systems exhibited axially lubricated flow and which systems exhibited viscous displacement. Volume fractions below 55% were not experimentally explored.

FIG. 3C is, in accordance with some embodiments, a temporal diagram of a cross section of the needle (needle inner diameter=304.8 μm) combined with a pressure versus time plot, highlighting the viscous displacement regime. This regime involved cyclic switching between a primary state where the viscous fluid filled the entire cross-section of the needle to a secondary state where the two fluids flowed as an intermittent axially lubricated flow. This resulted in a high and unstable pressure drop in the needle. The scale bar is 100 μm wide.

FIG. 3D is, in accordance with some embodiments, a temporal diagram of a cross section of the needle combined with a pressure versus time plot, highlighting the axially lubricated flow regime. The axially lubricated flow regime was stable over time and resulted in a much lower steady state pressure drop. The scale bar is 100 μm wide.

FIG. 4A is, in accordance with some embodiments, a plot of the pressure reduction coefficient (η) versus the volumetric flow rate of the outer fluid to that of the inner fluid (Qₒ/Qᵢ) for different viscosity ratios (λ).

FIG. 4B is, in accordance with some embodiments, a digital photograph of a needle (needle inner diameter=304.8 μm, scale bar is 100 μm wide) with eccentric inner and outer fluids, which resulted in a lower maximum pressure reduction coefficient than in systems with concentric flow.

FIG. 5A is, in accordance with some embodiments, an exploded view of a proof-of-concept double barreled syringe.

FIG. 5B is, in accordance with some embodiments, a photograph of a double barreled syringe.

FIG. 5C is a set of time-lapse images comparing the injectability of a high viscosity formulation through a commercial syringe (top) and a syringe configured and used in accordance with certain embodiments (bottom).

FIG. 5D shows a comparison of the force reduction coefficient of the overall double barreled syringe (rums), in accordance with certain embodiments, and the needle (η_{needle}) alone compared to corresponding control experiments in a comparator syringe-needle system.

FIG. 5E shows that an increase in concentration is possible for a nominal injection force of 25N by using a syringe configured and used according to certain embodiments described herein (e.g., a double barreled syringe).

FIG. 5F plots, in accordance with some embodiments, the injection force versus the concentration of monoclonal antibody concentration in a double barreled syringe.

FIG. 6A is an image showing, in accordance with some embodiments, axially lubricated flow in a needle connected to an axially lubricated flow injector.

FIG. 6B is a schematic illustration, in accordance with some embodiments, of an experimental setup used to measure the pressure reduction coefficient of the double barreled syringe.

FIG. 7 shows, in accordance with some embodiments, the contact angle measurement of HFE-7500 on a PTFE surface in an environment of a 26 cP glycerol/water mixture.

FIG. 8A shows, in accordance with certain embodiments, a plot of the timescale of convection/timescale of eccentricity (T_{c}/tₑ) versus the difference in densities of the inner fluid and outer fluid for different average volumetric flow rates (Q_{avg}).

FIG. 8B shows, in accordance with certain embodiments, a plot of the timescale of convection/timescale of eccentricity (T_{c}/tₑ) versus the orientation of the system (e.g., needle and/or chamber) when the difference in density between the inner fluid and outer fluid is 0.05 kg/m³ for different average volumetric flow rates (Q_{avg}).

FIG. 9 is, in accordance with certain embodiments, a schematic illustration of a droplet on a surface within a medium, which can be used to illustrate how the spreading coefficient is determined.

FIG. 10A is a cross-sectional view of an example of a needle with an inner fluid and outer fluid in concentric annular flow.

FIG. 10B is a cross-sectional view of an example of a needle with an inner fluid and outer fluid in fully eccentric annular flow.

FIG. 10C is a cross-sectional view of an example of a needle with an inner fluid and outer fluid in partially eccentric annular flow.

FIG. 11 plots, in accordance with certain embodiments, the capillary number of the inner fluid versus the capillary number of the outer fluid, and shows which systems exhibited axially lubricated flow and which systems exhibited viscous displacement.

FIG. 12A is, in accordance with certain embodiments, a top view schematic diagram of an interior surface of a needle comprising a texture.

FIG. 12B is, in accordance with certain embodiments, a three dimensional perspective of an interior surface of a needle comprising a texture.

### DETAILED DESCRIPTION

Disclosed herein are articles, systems, and methods for the injection of viscous fluids. For example, inventive articles, systems, and methods for injecting viscous fluids, such as concentrated drug formulations, via lubrication are described. In some embodiments, injectability of an inner fluid, such as a concentrated drug formulation, is desired. However, the non-linear relationship between formulation concentration and viscosity can greatly limit the ability to inject high concentration drug formulations, which are frequently needed for biologics and/or subcutaneous administration. As drug concentrations increase over 50 mg/mL, the corresponding viscosities frequently range from 20 cP to 1000 cP, making injection through conventional delivery methods (e.g., syringes) extremely challenging. For example, high hydraulic resistance presented by flow through needles at such high concentrations frequently induces large back pressures. In some embodiments, the articles, systems, and/or methods described herein reduce these resistances and enhance the injectability of such high concentration drug formulations, and other high viscosity fluids, by achieving axially lubricated flow with the fluid of interest (e.g., the inner fluid) and a lubricating fluid (e.g., the outer fluid).

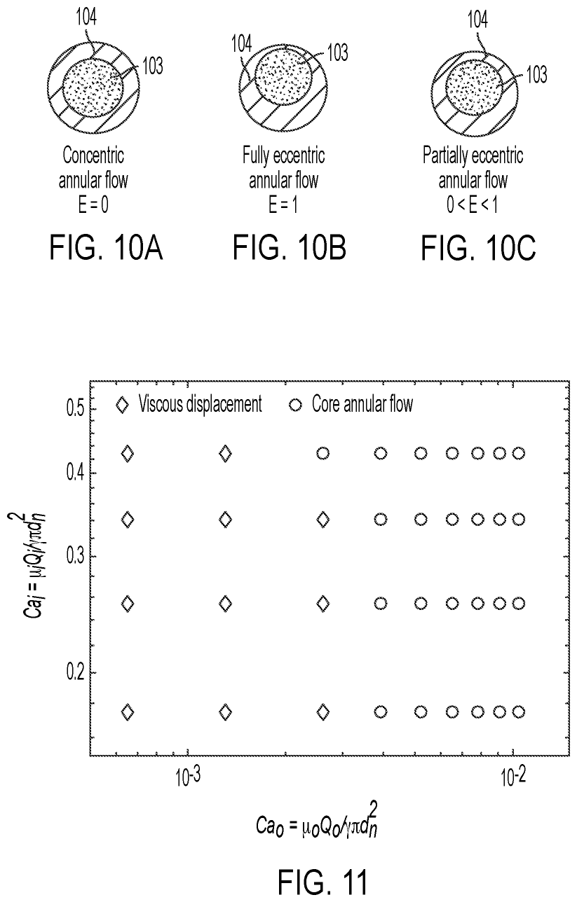

However, axially lubricated flow can be very difficult to achieve in practical systems. For example, eccentricity (e.g., as shown in FIGS. 10B and 10C, compared to a concentric system in FIG. 10A) frequently arises if the densities of the inner fluid and outer fluid are not substantially the same, such that the inner fluid contacts the interior surface of the needle and/or chamber, reducing the lubrication effect from the outer fluid. However, trying to match the densities of the inner and outer fluids can be extremely impractical in many cases. Avoiding eccentricity can be especially difficult in cases where the outer fluid and inner fluid are miscible. While vertical operation could be used to avoid eccentricity in certain cases, this is also typically impractical, as most subcutaneous injections are not administered vertically. Moreover, vertical operation would only facilitate injection of miscible inner and outer fluids, in certain cases, and would typically not work with immiscible fluids. Certain of the embodiments disclosed herein are capable of achieving axially lubricated flow in practical systems, despite these challenges.

In certain embodiments, the articles, systems, and/or methods comprise an outer fluid axially surrounding the inner fluid. In certain cases, the outer fluid preferentially wets the interior surface of a needle and/or chamber through which the fluid flows, relative to the inner fluid, which helps ensure that the inner fluid does not contact the interior surface of the needle and/or chamber, even in cases where eccentricity of the fluid flow is high, and even in cases where the needle is close to horizontal during administration. In some embodiments, the interior surface of the needle is textured to facilitate preferential wetting by the outer fluid. In some cases, the interior surface of the needle is coated to facilitate preferential wetting by the outer fluid.

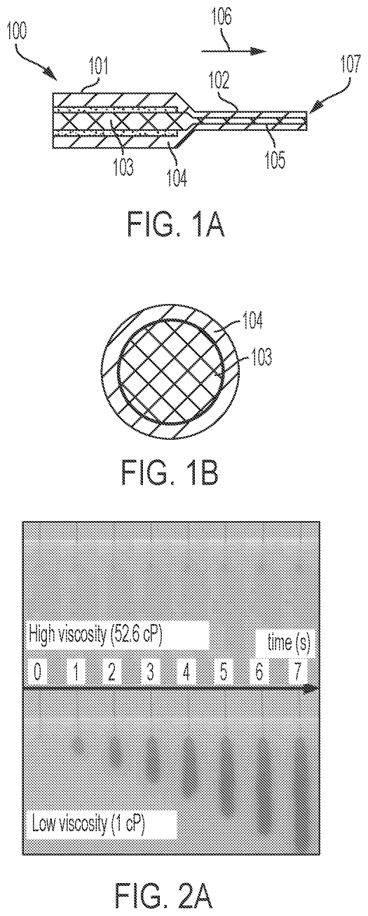

Articles for delivery of a fluid are described herein. One such article is illustrated schematically in FIGS. 1A-1B. In some embodiments, the article comprises a chamber. For example, in accordance with some embodiments, article 100 in FIG. 1A comprises chamber 101. In some embodiments, the diameter of the chamber is greater than the diameter of the needle. In some embodiments, the chamber comprises a biocompatible material. In some embodiments, the material of the chamber is selected such that when the inner fluid and outer fluid are in contact with each other and with the chamber, the outer fluid preferentially wets an interior surface of the chamber relative to the inner fluid.

In certain embodiments, the article comprises a needle. For example, in accordance with certain embodiments, article 100 in FIG. 1A comprises needle 102. In some embodiments, the article is a syringe needle system. In certain embodiments, the article comprises a plurality of needles. For example, in some cases, the article comprises greater than or equal to 1, greater than or equal to 2, greater than or equal to 3, greater than or equal to 10, greater than or equal to 50, or greater than or equal to 100 needles. In certain instances, the articles comprises less than or equal to 1,000, less than or equal to 500, less than or equal to 100, less than or equal to 50, less than or equal to 10, or less than or equal to 5 needles. Combinations of these ranges are also possible (e.g., 1-1,000).

In accordance with some embodiments, the article comprises a microneedle patch. In some embodiments, the microneedle patch comprises an array of needles, optionally arranged in a periodic pattern. In some such embodiments, the inner and outer fluids can be delivered to a subject (e.g., a patient) via the needles of the microneedle patch

In some embodiments the article is manually actuated. For example, in certain embodiments, injection of the inner fluid can be achieved by applying pressure by hand. Manual actuation is not required, however, and in some embodiments, the article is non-manually actuated. For example, in some embodiments, the article is actuated by a mechanical spring and/or an electrical motor.

In accordance with some embodiments, the needle is fluidically connected to the chamber. For example, in the example embodiment shown in FIG. 1A, needle 102 is fluidically connected to chamber 101. The needle may be directly connected to the chamber (e.g., with nothing in between) or it may be indirectly connected to the chamber (e.g., with an additional chamber in between). According to some embodiments, the chamber is upstream of the needle, such that fluid in the chamber could flow and/or be transported to the needle. For example, in the example shown in FIG. 1A, chamber 101 is upstream of needle 102, such that fluid in chamber 101 can flow from chamber 101 to needle 102.

In some embodiments, the article comprises an inner fluid. For example, in accordance with certain embodiments, article 100 in FIG. 1A comprises inner fluid 103. In accordance with some embodiments, the inner fluid extends from the chamber into the needle. For example, as illustrated in FIG. 1A, inner fluid 103 extends from chamber 101 into needle 102. In certain embodiments, the inner fluid flows through the needle. For example, as shown in FIG. 1A, inner fluid 103 flows through needle 102 in the direction of arrow 106.

In certain embodiments, the article comprises an outer fluid. For example, in some instances, article 100 in FIG. 1A comprises outer fluid 104. In accordance with certain embodiments, the outer fluid extends from the chamber into the needle. For example, as illustrated in FIG. 1A, outer fluid 104 extends from chamber 101 into needle 102. In some embodiments, the outer fluid axially surrounds the inner fluid, as described in more detail below. According to some embodiments, the outer fluid flows through the needle. For example, as shown in FIG. 1A, outer fluid 104 flows through needle 102 in the direction of arrow 106.

Examples of fluids include liquids, such as pure liquids and mixtures of liquids, as well as liquids combined with non-liquids, such as liquid/gas mixtures and liquid/solid mixtures, such as suspensions.

According to certain embodiments, the article is configured such that, when an inner fluid and an outer fluid are transported through the needle, the outer fluid axially surrounds the inner fluid. A first fluid is said to “axially surround” a second fluid when a continuous pathway can be traced, within the first fluid, that surrounds the longitudinal axis of the second fluid. For example, as illustrated in FIGS. 1A-1B, outer fluid 104 axially surrounds inner fluid 103. In some embodiments, the outer fluid is positioned around the circumference of the inner fluid, but does not surround the inner fluid at the end of the stream exiting the needle (or other fluidic pathway). In the non-limiting example shown in FIG. 1A, for example, outer fluid 104 is positioned around the circumference of inner fluid 103 but does not surround inner fluid 103 at point 107 (the end of needle 105). In certain embodiments, the outer fluid can axially surround the inner fluid such that the inner fluid is elongated, for example, having a ratio of length to cross-sectional dimension of at least 5:1, at least 10:1, at least 25:1, or greater.

According to some embodiments, the outer fluid preferentially wets an interior surface of the needle and/or the chamber relative to the inner fluid. For example, referring to the example shown in FIG. 1A, in certain embodiments, outer fluid 104 preferentially wets interior surface 105 of needle 102 relative to inner fluid 103.

In some embodiments, the outer fluid preferentially wets an interior surface of the needle relative to the inner fluid when for the inner fluid, the outer fluid, and the interior surface of the needle, the spreading coefficient (Sₒₙ₍ᵢ₎) is greater than or equal to 0. FIG. 9 is a schematic illustration of a droplet of the outer fluid on the interior surface of the needle, where the outer droplet is surrounded by the inner fluid. The spreading coefficient can be determined according to the following equations:

$${S}_{o n ( i )} = {γ}_{n i} - ( {γ}_{n o} + {γ}_{o i} ) \qquad ( Equation 1 )$$

$$cos ( {θ}_{o n ( i )} ) = \frac{{γ}_{n i} - {γ}_{n o}}{{γ}_{o i}} \qquad ( Equation 2 )$$

$${S}_{o n ( i )} = {γ}_{o i} ( cos ( {θ}_{o n ( i )} ) - 1 ) \qquad ( Equation 3 )$$

In the equations above, gamma (γ) is the surface tensions of the various interfaces involved, where n is the subscript for an interior surface of the needle, o is the subscript for the outer fluid, and i is the subscript for the inner fluid. For example, γₙᵢ denotes the surface tension between the needle and the inner fluid, γₙₒ denotes the surface tension between the needle and the outer fluid, and γₒᵢ denotes the surface tension between the outer fluid and the inner fluid. For example, in some embodiments, cos(θₒₙ₍ᵢ₎) and γₒᵢ are measured, and the spreading coefficient is determined by Equation 3. The spreading coefficient is specific to the three components (e.g., the interior surface of the needle, the inner fluid, and the outer fluid).

In certain embodiments, the inner fluid does not contact an interior surface of the needle. For example, in some embodiments, inner fluid 103 in FIG. 1A does not contact interior surface 105 of needle 102. According to some embodiments, the inner fluid does not contact an interior surface of the needle for a period of time. For example, in some cases, the period of time is between initiating flow of the inner fluid and/or outer fluid and ejection of the inner fluid and/or outer fluid from the needle. In certain cases, the period of time is at least a portion of time (e.g., at least 50%, at least 75%, at least 90%, or the entirety of the time) between initiating flow of the fluid and ejection of fluid from the needle.

According to some embodiments, the inner fluid comprises a drug, a monoclonal antibody, an enzyme, a peptide, a recombinant therapeutic protein, a biologic, a bone putty, a hydrogel, cells, and/or a biopharmaceutical. For example, in certain embodiments, the inner fluid comprises a concentrated drug formulation (e.g., biologic).

According to certain embodiments, the outer fluid has a lower viscosity than the inner fluid. In some embodiments, the ratio of the viscosity of the inner fluid to the viscosity of the outer fluid (μᵢ/μₒ)>1. In some embodiments, the ratio of the viscosity of the inner fluid to the viscosity of the outer fluid (μᵢ/μₒ) is greater than or equal to 3, greater than or equal to 5, greater than or equal to 8, or greater than or equal to 10.

In some cases, the outer fluid comprises water, a buffer (e.g., a pharmaceutically acceptable buffer, such as a buffer used in a pharmaceutical product, such as a biologic), a formulation (e.g., a pharmaceutical formulation, such as a biologic formulation), a water-based solution, saline, a biocompatible oil (e.g., squalene, a fluorinated oil (e.g., HFE-7500), mineral oil, and/or triglyceride oil), benzyl benzoate, a metabolizable oil, an immunologic adjuvant (e.g., MF59, AS02, AS03 and/or AS04), and/or safflower oil.

In some embodiments, the outer fluid and inner fluid are immiscible. For example, according to certain embodiments, neither the outer fluid nor the inner fluid is soluble in the other in an amount of more than 0.001 mass fraction, more than 0.0001 mass fraction, or more than 0.00001 mass fraction. In certain embodiments, the outer fluid and inner fluid are immiscible at the temperature at which the fluids are flowed. In some cases, the outer fluid and inner fluid are immiscible at 25° C.

The use of immiscible inner fluids and outer fluids is not necessarily required, and in some embodiments, the outer fluid and inner fluid are miscible. For example, according to some embodiments, the outer fluid and/or the inner fluid is soluble in the other in an amount of more than 0.001 mass fraction, more than 0.01 mass fraction, or more than 0.1 mass fraction. In certain embodiments, the outer fluid and inner fluid are miscible at the temperature at which the fluids are flowed. In some cases, the outer fluid and inner fluid are miscible at 25° C.

For the systems and methods described herein, the timescale of convection (T_{c}) is how long the inner fluid and outer fluid take to travel through the system (e.g., the needle and/or the chamber) while they are in direct contact with each other. The timescale of convection is calculated by estimating the average volumetric flow rate of the multi-fluid system. Specifically, the average volumetric flowrate and timescale of convention are calculated using the following equations:

$${Q}_{a v g} = \frac{{Q}_{i} + {Q}_{O}}{2} \qquad ( Equation 4 )$$

$${T}_{c} = \frac{L}{\overline{V}} = \frac{L {A}_{c}}{{Q}_{a v g}} \qquad ( Equation 5 )$$

Where Q_{avg} is the average flowrate of the inner and outer fluids, Qi is the volumetric flowrate of the inner fluid, Qo is the volumetric flowrate of the outer fluid, L is the length of the system, A_{c} is the cross-sectional area of the system, and V is the average linear velocity.

For the systems and methods described herein, the timescale of eccentricity (tₑ) is the time for spatially stable eccentricity to arise in any part of the system (e.g., the needle and/or the chamber) comprising the inner fluid and outer fluid. Timescale of eccentricity may be measured according to the following equation:

$${t}_{e} = \sqrt{\frac{2 s}{\vert g cos ( θ ) ( 1 - \frac{{ρ}_{i}}{{ρ}_{o}} ) \vert}} \qquad ( Equation 6 )$$

Where θ is the angle between the length of the needle and the horizontal plane, ρᵢ is density of the inner fluid, g is the gravitational constant and s is the radial displacement of the centerline of the inner fluid from the axial centerline of the device, and ρₒ is density of the outer fluid.

In certain embodiments, the timescale of convection (T_{c}) is less than the timescale of eccentricity (tₑ). For example, in some embodiments, the ratio of the timescale of convection (T_{c}) for the inner fluid and outer fluid to the timescale of eccentricity (tₑ) for the inner fluid and outer fluid is less than or equal to 1, less than or equal to 0.75, less than or equal to 0.5, or less than or equal to 0.1. In some embodiments, when the timescale of convection (T_{c}) is less than the timescale of eccentricity (tₑ), the fluids do not substantially exhibit eccentricity while in the system (e.g., the needle and/or chamber).

For the systems and methods described herein, the timescale of mixing (tₘ) is the time needed for 50% of the outer fluid to mix with the inner fluid as they travel through the system or a portion thereof (e.g., the needle and/or the chamber) while they are in direct contact with each other. The timescale of mixing may be calculated using the following equation:

$${t}_{m} = \frac{{l}_{d}^{2}}{D i} \qquad ( Equation 7 )$$

Where Dᵢ is the diffusion coefficient of one or more components of the inner fluid (e.g., a drug (e.g., a biologic) in the inner fluid) in the outer fluid and ld is the diameter of the part of the system (e.g., the needle and/or the chamber) where the fluids are in direct contact with each other. In embodiments where the system has portions with different diameters (e.g., a system comprising a chamber and a needle where the chamber has a larger diameter than the needle), the timescale of mixing may be determined using Equation 7 for each portion individually. In embodiments where the system has a varying geometry (e.g., if the chamber had an oval shape), the timescale of mixing may be determined using Equation 7 in conjunction with an integral approach.

In certain embodiments, the timescale of convection (T_{c}) is less than the timescale of mixing (tₘ) in one or more portions of the system (e.g., in the needle and/or in the chamber) or in the entire system. For example, in some embodiments, the timescale of convection is less than the timescale of mixing in the needle and/or the timescale of convection is less than the timescale of mixing in the chamber. For example, in some embodiments, the ratio of the timescale of convection (T_{c}) for the inner fluid and outer fluid to the timescale of mixing (tₘ) for the inner fluid and outer fluid is less than or equal to 1, less than or equal to 0.75, less than or equal to 0.5, less than or equal to 0.1 or less than or equal to 0.01. In some embodiments, when the timescale of convection (T_{c}) is less than the timescale of mixing (tₘ), the fluids do not substantially mix while in the system or a portion thereof (e.g., the needle and/or chamber).

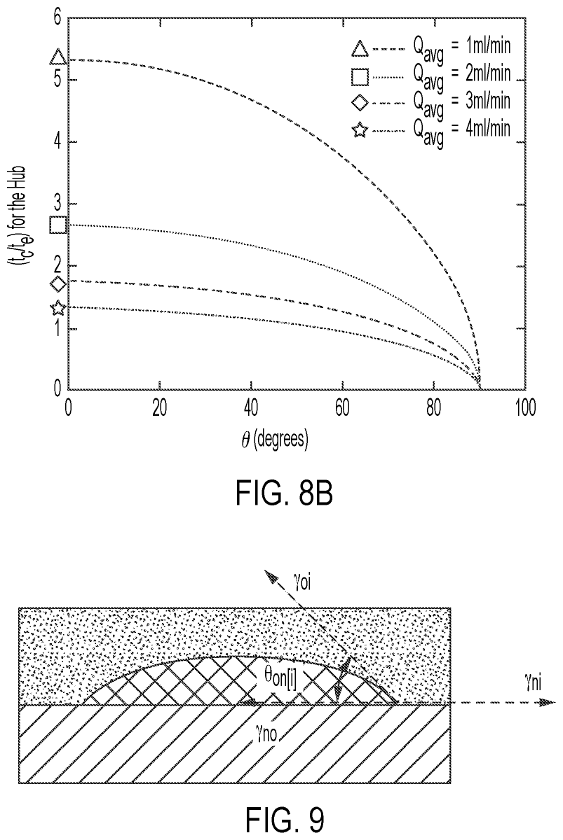

In some embodiments, the densities of the inner and outer fluids and/or the volumetric flow rate (Q) affects the timescale of convection and/or the ratio of the timescale of convection to the timescale of eccentricity. For example, FIG. 8A demonstrates that, in accordance with certain embodiments, a t_{c}/tₑ of less than or equal to 1 is easier to achieve with smaller differences in density between the inner fluid and outer fluid and/or with a higher average volumetric flow rate of the inner fluid (Qᵢ).

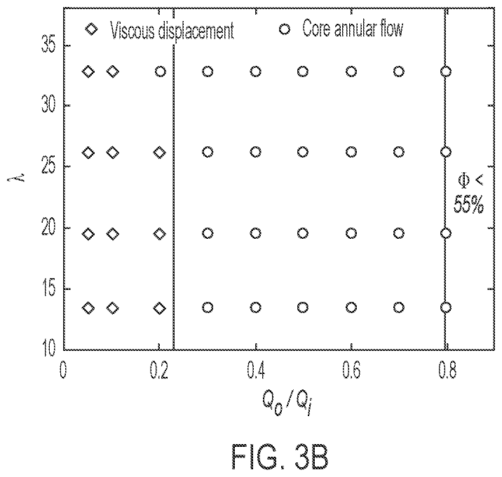

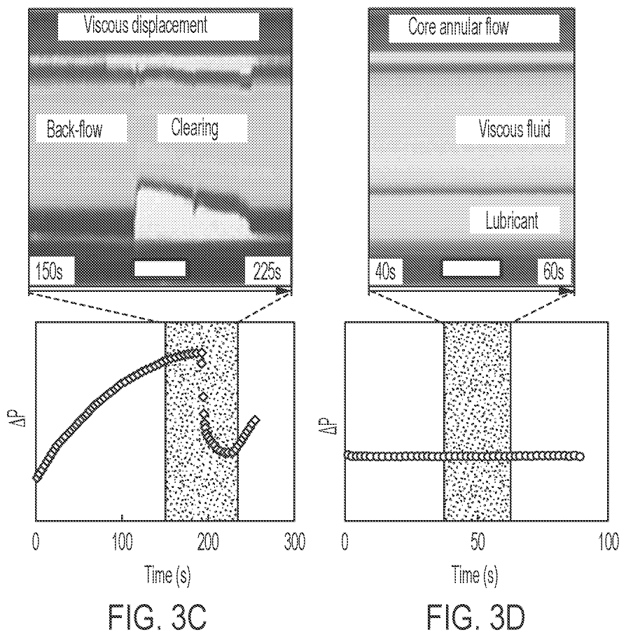

In certain embodiments, when the outer fluid flow rate is too low compared to the inner fluid flow rate, a viscous displacement regime is observed rather than an axially lubricated flow regime. In a viscous displacement regime, the outer fluid fills the entire cross-section of the needle and forces both the inner fluid and the outer fluid to back-flow into the outer fluid inlet. However, in certain cases, the backflow cannot be sustained due to the constant mass flux that is imposed on the outer fluid, resulting in a sudden overflow of the outer fluid into the needle. In some instances, this flow decreases until it is completely hindered once again, and the process repeats. In certain embodiments, this cyclic behavior (as shown in FIG. 3C) results in unsteady and significantly worse lubrication compared to an axially lubricated flow regime (as shown in FIG. 3D).

In accordance with some embodiments, the ratio of the volumetric flow rate of the outer fluid (Qₒ) to the volumetric flow rate of the inner fluid (Qᵢ) is greater than 0.1. In some embodiments, the ratio of the volumetric flow rate of the outer fluid (Qₒ) to the volumetric flow rate of the inner fluid (Qᵢ) is greater than or equal to 0.2, greater than or equal to 0.4, or greater than or equal to 0.6. In certain embodiments, the ratio of the volumetric flow rate of the outer fluid (Qₒ) to the volumetric flow rate of the inner fluid (Qᵢ) is less than or equal to 1.

In some embodiments, the outer fluid and inner fluid do not mix substantially in the needle and/or chamber, because mixing dilutes the inner fluid, reducing the benefits of axially lubricated flow. In certain embodiments, the timescale of convection is shorter than the time it takes for the inner fluid and outer fluid to mix substantially in the needle and/or chamber. In accordance with some embodiments, the outer fluid mixes with the inner fluid at most 50% while in the needle and/or chamber. That is, at most 50% of the outer fluid is mixed with the inner fluid while in the needle and/or chamber while the remainder of the outer fluid remains unmixed with the inner fluid. For example, in certain embodiments, the outer fluid mixes with the inner fluid at most 40%, at most 30%, at most 20%, or at most 10% while in the needle and/or chamber. According to certain embodiments, the percentage of mixing can be determined by visual inspection. In some embodiments, this could be accomplished by dyeing the inner fluid and/or outer fluid, taking photographs at the outlet of the needle, and measuring the extent of mixing of the two fluids from the diffusion and/or spreading of the dye(s). In certain embodiments, the extent of mixing could be measured at different lengths by cutting the needle to the length of interest, and photographing the fluids at the outlet.

In some embodiments, the inner fluid and the outer fluid comprise completely different components. For example, in some embodiments, the inner fluid and the outer fluid do not have any components in common. One such example would be if the inner fluid comprises a drug and water, while the outer fluid comprises an organic solvent.

In some embodiments, the inner fluid and the outer fluid comprise one or more components (e.g., a solvent and/or a buffer) that are the same. For example, in certain embodiments, the inner fluid and the outer fluid both comprise water.

In certain embodiments, the inner fluid and/or the outer fluid comprises one or more components that are different. For example, in some embodiments, the inner fluid comprises water and the outer fluid does not.

In certain embodiments, the inner fluid and the outer fluid comprise one or more components that are different and one or more components that are the same. For example, in some embodiments, the inner fluid and the outer fluid comprise the same components except that the inner fluid also has a drug (e.g., a biologic). For example, in certain embodiments, the inner fluid and the outer fluid both comprise water, but the inner fluid has a drug (e.g., a biologic) and the outer fluid does not. In some embodiments, the inner fluid and the outer fluid comprise exactly the same components (e.g., a buffer) except that one of the fluids (e.g., the inner fluid) has an additional component (e.g., a drug).

In some embodiments, the inner fluid and the outer fluid comprise exactly the same components (e.g., a buffer and a drug), but the concentrations of one or more of the components are different (e.g., a drug). For example, in some embodiments, the inner fluid and the outer fluid comprise exactly the same components (e.g., a buffer and a drug), but the concentration of one or more of the components (e.g., a drug) is higher in the inner fluid. As a skilled person would understand, in some embodiments, the different concentration of one or more of the components could result in different physical and/or chemical properties. For example, in an embodiment where the inner fluid has a high concentration of a biologic drug and the outer fluid has a low concentration of the biologic drug, but the inner and outer fluids are otherwise identical, the viscosity and/or density of the inner fluid may be much higher than that of the outer fluid.

In some embodiments, the molar concentration of one component (e.g., a drug) in the outer fluid is greater than or equal to 5%, greater than or equal to 10%, greater than or equal to 20%, greater than or equal to 30%, greater than or equal to 40%, greater than or equal to 50%, greater than or equal to 60%, greater than or equal to 75%, greater than or equal to 90%, or greater than or equal to 95% less than the molar concentration of that component in the inner fluid. In some embodiments, the molar concentration of one component (e.g., a drug) in the outer fluid is less than or equal to 100%, less than or equal to 99%, less than or equal to 95%, less than or equal to 90%, less than or equal to 80%, less than or equal to 70%, less than or equal to 60%, or less than or equal to 50% the molar concentration of that component in the inner fluid. Combinations of these ranges are also possible (e.g., greater than or equal to 5% and less than or equal to 100%, or greater than or equal to 10% and less than or equal to 50%). For example, if the molar concentration of the component was 1M in the inner fluid and 0.1M in the outer fluid, the molar concentration of the component in the outer fluid would be 90% less than that in the inner fluid.

When the inner fluid and the outer fluid are in concentric contact and are moving, one or more components of the inner fluid (e.g., a drug, such as a biologic) may begin to diffuse into the outer fluid. The radial position of the distinction between the inner and outer fluids (R(x)) is given by the following equation:

$$R ( x ) = {R}_{0} + \sqrt{\frac{D x}{\overline{V}}} \qquad ( Equation 8 )$$

Where R₀ is the radius of the inner fluid at the beginning of any section of interest where the fluids are in contact, x is the axial position along the section, D is the diffusion coefficient of the component (e.g., a drug) in the outer fluid, and V̅ is the average velocity of the inner fluid. The extent of this diffusion can be validated by visualization as described elsewhere herein (e.g., by using dye molecules with the same diffusion coefficient as the component (e.g., drug)). As used herein, the radial position of the distinction between the inner and outer fluid (R(x)) means the distance between the center of the inner fluid and the distinction (e.g., boundary) between the inner and outer fluids. For example, when the inner fluid and the outer fluid first make contact and no diffusion has taken place, R(x) will be the same as R₀. However, as the fluids move through the system and the axial position (x) increases, R(x) will become larger than R₀, in some embodiments.

According to some embodiments, the outer fluid is a Newtonian fluid. For example, in accordance with certain embodiments, the viscous stresses arising from flow of the outer fluid at every point is linearly related to the local strain rate. Examples of suitable Newtonian fluids include water, a water-based solution, a buffer (e.g., a pharmaceutically acceptable buffer, such as a buffer used in a pharmaceutical product, such as a biologic), a formulation (e.g., a pharmaceutical formulation, such as a biologic formulation), saline, a biocompatible oil (e.g., squalene, a fluorinated oil (e.g., HFE-7500), mineral oil, and/or triglyceride oil), benzyl benzoate, a Metabolizable oil, an immunologic adjuvant (e.g., MF59, AS02, AS03 and/or AS04), and/or safflower oil.

In accordance with certain embodiments, the outer fluid is a yield stress fluid. For example, according to some embodiments, the outer fluid deforms and/or flows only when subjected to a stress above a certain critical value specific to the yield stress fluid. Examples of suitable yield stress fluids include bone putty, hydrogels, hydrogel microbeads, and/or polymer solutions (example: polyethylene glycol).

In certain embodiments, there is an additional fluid. In some embodiments, the additional fluid is an additional lubricating layer. In certain embodiments, the outer fluid and/or the additional fluid comprise a surfactant. In some cases, the surfactant reduces and/or prevents coalescence and/or breakdown. In certain instances, the additional fluid is more biocompatible than the outer fluid. In some embodiments, the use of the additional fluid results in enhanced biocompatibility. In certain embodiments, the additional fluid (e.g., an additional fluid comprising a surfactant) increases the spreading coefficient. In some embodiments, the additional fluid (e.g., an additional fluid comprising a surfactant) increases the capillary number of the inner fluid and/or outer fluid.

In some embodiments, the needle comprises an interior surface. For example, in some instances, needle 102 in FIG. 1A comprises interior surface 105.

In certain embodiments, the interior surface of the needle comprises a texture. For example, in some embodiments, the interior surface of the needle comprises a plurality of features. For example, in certain embodiments, the external surface of the conduit comprises milliscale, microscale, and/or nanoscale features. The texture may be used, in certain embodiments, to control the wettability of the surface. Any of a variety of features may be used. Non-limiting examples of protrusions include spherical or hemispherical protrusions. In some embodiments, the features comprise protrusions such as ridges, spikes, and/or posts. The features may be formed, for example, by etching away or otherwise removing material from which the surface is made, in some embodiments. In other embodiments, the features may be added to the surface (e.g., by depositing the features onto the interior surface of the needle and/or chamber, for example). The features may be made of material that is the same as or different from the material from which the interior surface is made. In certain embodiments, the features may be dispersed on the interior surface in a random (e.g., fractal) or patterned manner.

According to some embodiments, the maximum height of the milliscale features is greater than 100 micrometers and up to 1 millimeter, greater than 100 micrometers and up to 200 micrometers, from 200 micrometers to 300 micrometers, from 300 micrometers to 500 micrometers, from 500 micrometers to 700 micrometers, from 700 micrometers to 1 millimeter, from 1 millimeter to 3 millimeters, from 3 millimeters to 5 millimeters, and/or from 5 millimeters to 10 millimeters. Combinations of the above cited ranges are also possible (e.g., from 300 micrometers to 700 micrometers, or from 200 micrometers to 1 millimeter).

According to some embodiments, the maximum height of the microscale features is from 1 micrometer to 10 micrometers, 10 micrometers to 20 micrometers, 20 micrometers to 30 micrometers, 30 micrometers to 50 micrometers, 50 micrometers to 70 micrometers, or 70 micrometers to 100 micrometers. Combinations of the above cited ranges are also possible (e.g., 30 micrometers to 70 micrometers, or 20 micrometers to 100 micrometers).

According to some embodiments, the maximum height of the nanoscale features is from 1 nm to 100 nm, 100 nm to 200 nm, 200 nm to 300 nm, 300 nm to 500 nm, 500 nm to 700 nm, or 700 nm to 1 micrometer. Combinations of the above cited ranges are also possible (e.g., 300 nm to 700 nm, or 200 nm to 1 micrometer).

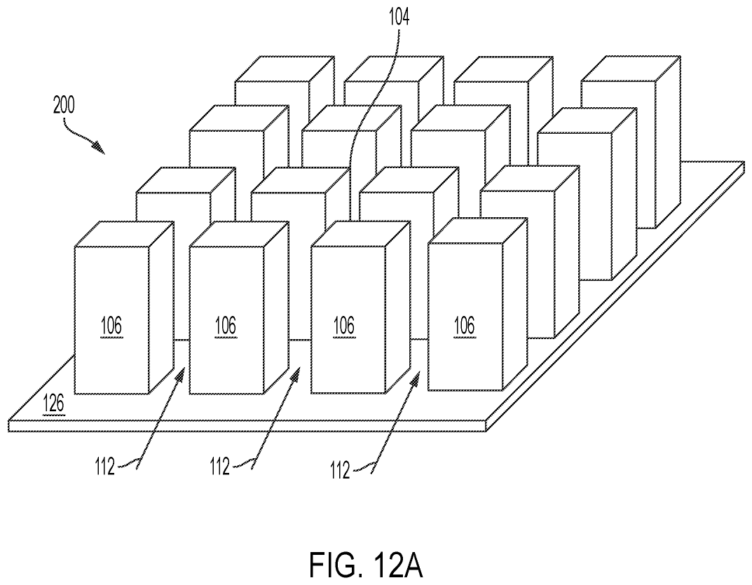

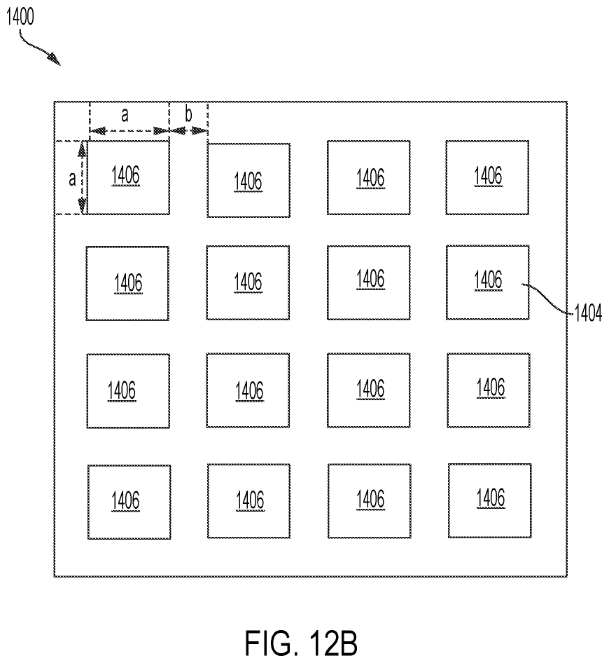

According to certain embodiments, the features (e.g., the milliscale, microscale, and/or nanoscale features) are distributed over the interior surface of the needle and/or the chamber such that the features occupy a particular solid fraction of the interior surface. The term “solid fraction” (also referred to as φₛ) occupied by a plurality of features on a surface, as used herein, refers to the area fraction of the surface that is occupied by the features. The solid fraction can be calculated by dividing the sum of the areas that the features occupy on the interior surface by the geometric surface area of the interior surface over which those features are distributed. For example, referring to FIGS. 12A-12B, interior surface portion 1400 (e.g., a portion of the interior surface of the needle) comprises a plurality of features 1406. Features 1406 in FIGS. 12A-12B are squares with side lengths a, and thus, each occupies an area on the interior surface equal to a². The remaining area of the interior surface is not occupied by features. In the set of embodiments illustrated in FIGS. 12A-12B, each of features 1406 have identical side lengths a and identical nearest neighbor spacings b. Accordingly, the surface solid fraction (φₛ) occupied by the features in FIGS. 12A-12B would be calculated as follows:

φs=a²/(a+b)² (Equation 9)

In certain embodiments, the interior surface of the needle comprises a texture for which the solid fraction (cps) is less than or equal to 0.5. In some embodiments, the interior surface of the needle comprises a texture for which the solid fraction (cps) is less than or equal to 0.25 or less than or equal to 0.1.

In certain embodiments, the interior surface of the chamber comprises a texture for which the solid fraction (φₛ) is less than or equal to 0.5. In some embodiments, the interior surface of the needle comprises a texture for which the solid fraction (φₛ) is less than or equal to 0.25 or less than or equal to 0.1.

In certain embodiments, a third fluid (in addition to the inner fluid and the outer fluid) can be impregnated between the features on the interior surface of the needle and/or chamber. The third fluid may, in some embodiments, be stably contained between the features such that the third fluid remains contained between the features while the inner and outer fluids are transported through the needle (and/or the chamber). The third fluid can be stably contained between the features, for example, by spacing the features sufficiently close such that the third liquid is stably contained between the features (e.g., via surface tension forces). In certain embodiments, the third fluid is contained between the features but does not cover the tops of the features. In some embodiments, the properties of the third fluid may be tailored to control the wettability of the interior surface of the needle and/or chamber.

In accordance with some embodiments, for a given inner fluid, outer fluid, and interior textured surface of the needle and/or chamber, the spreading coefficient (Sₒₙ₍ᵢ₎) is greater than or equal to 0. In some embodiments, the texture imparts wettability for at least one fluid (e.g., the outer fluid) when a droplet of that fluid is present on the interior surface of the needle in another fluid (e.g., the inner fluid). That is, in certain instances, the at least one fluid (e.g., the outer fluid) is wetting when the texture is present, but would not be wetting in an identical system without the texture.

According to certain embodiments, the interior surface of the needle comprises a coating. For example, in some embodiments, the interior surface of the needle comprises a conformal, smooth coating with limited discontinuities. In some embodiments, a conformal, smooth coating with limited discontinuities has less than or equal to 10⁸, less than or equal to 10⁶, or less than or equal to 10⁴ discontinuities/m². A coating is considered to be conformal if 90% of the facial area of the coating is within 20% of the average thickness of the coating. In accordance with some embodiments, for the inner fluid, the outer fluid, and the interior surface of the coating, the spreading coefficient (Sₒₙ₍ᵢ₎) is greater than or equal to 0. In some embodiments, the coating imparts wettability for at least one fluid (e.g., the outer fluid) when a droplet of that fluid is present on the interior surface of the needle in the other fluid (e.g., inner fluid). That is, in certain instances, the at least one fluid (e.g., the outer fluid) is wetting when the texture is present, but would not be wetting in an identical system without the texture.

The needle can have, in accordance with certain embodiments, any of a variety of lengths. Certain of the embodiments described herein can be used to achieve stable core sheath flow within a needle having a relatively long length. According to certain embodiments, the needle has a length of greater than or equal to 5 microns, greater than or equal to 10 microns, greater than or equal to 25 microns, greater than or equal to 50 microns, greater than or equal to 100 microns, greater than or equal to 1 mm, greater than or equal to 5 mm, greater than or equal to 10 mm, or greater than or equal to 100 mm. According to some embodiments, the needle has a length of less than or equal to 250 mm, less than or equal to 100 mm, less than or equal to 50 mm, less than or equal to 10 mm, less than or equal to 5 mm, less than or equal to 1 mm, less than or equal to 500 microns, less than or equal to 100 microns, less than or equal to 50 microns, or less than or equal to 25 microns. Combinations of these ranges are also possible (e.g., 5 microns to 5 mm or 5 mm to 10 mm).

It should be understood that the use of relatively long needles is not required, and that in other embodiments, the needle is relatively short. For example, in some embodiments, the needle has a length of less than 5 mm, less than or equal to 1 mm, less than or equal to 500 microns, or less than or equal to 100 microns.

In certain embodiments, the needle is narrow. For example, in some cases, the needle has an inner diameter of greater than or equal to 5 microns, greater than or equal to 10 microns, greater than or equal to 25 microns, greater than or equal to 50 microns, greater than or equal to 100 microns, greater than or equal to 250 microns, greater than or equal to 500 microns, or greater than or equal to 750 microns. In some embodiments, the needle has an inner diameter of less than or equal to 1 mm, less than or equal to 750 microns, less than or equal to 500 microns, less than or equal to 310 microns, less than or equal to 250 microns, less than or equal to 100 microns, less than or equal to 50 microns, less than or equal to 25 microns, or less than or equal to 10 microns. Combinations of these ranges are also possible (e.g., greater than or equal 5 microns and less than or equal to 1 mm, or greater than or equal to 10 microns and less than or equal to 310 microns).

Methods are also described herein. In some embodiments, the method comprises initiating flow of at least a portion (e.g., at least 50%, at least 75%, at least 90%, or all) of an inner fluid (e.g., an inner fluid described herein) within an article described herein. According to some embodiments, at least a portion (e.g., at least 50%, at least 75%, at least 90%, or all) of the inner fluid is transported from the chamber to the needle. In certain embodiments, at least a portion (e.g., at least 50%, at least 75%, at least 90%, or all) of the inner fluid is ejected from the needle. For example, in some embodiments, at least a portion of inner fluid 103 in FIG. 1A is transported from chamber 101 to needle 102, and is ejected from needle 102.

In certain embodiments, the method comprises initiating flow of a least a portion (e.g., at least 50%, at least 75%, at least 90%, or all) of an outer fluid (e.g., an outer fluid described herein) within an article described herein. According to some embodiments, at least a portion (e.g., at least 50%, at least 75%, at least 90%, or all) of the outer fluid is transported from the chamber to the needle. In certain embodiments, at least a portion (e.g., at least 50%, at least 75%, at least 90%, or all) of the outer fluid is ejected from the needle. For example, in some embodiments, at least a portion of outer fluid 104 in FIG. 1A is transported from chamber 101 to needle 102, and is ejected from needle 102.

In some embodiments, it is beneficial for lower amounts of the outer fluid to be ejected compared to the amount of inner fluid ejected (e.g., such that a patient is not exposed to large amounts of a lubricating fluid). According to certain embodiments, the ratio of a volume of the inner fluid ejected from the needle to the total volume (e.g., inner fluid and outer fluid) ejected from the needle (Φ) (the volume fraction) is greater than or equal to 0.5, greater than or equal to 0.6, greater than or equal to 0.7, greater than or equal to 0.8, or greater than or equal to 0.9. The volume fraction (Φ) can also be expressed as:

Φ=Qᵢ/(Qᵢ+Qₒ) (Equation 10)

In accordance with some embodiments, when the inner fluid has a certain capillary number and the outer fluid has a certain capillary number, axially lubricated flow may be observed, whereas viscous displacement may otherwise be observed. For example, FIG. 11 plots, in accordance with certain embodiments, the capillary number of the inner fluid versus the capillary number of the outer fluid, and shows which systems exhibited axially lubricated flow and which systems exhibited viscous displacement.

According to certain embodiments, the capillary number of the inner fluid is greater than or equal to 0.01, greater than or equal to 0.1, greater than or equal to 1, greater than or equal to 10, greater than or equal to 20, or greater than or equal to 25. In some embodiments, the capillary number of the inner fluid is less than or equal to 30, less than or equal to 25, less than or equal to 10, less than or equal to 1, or less than or equal to 0.1. Combinations of these ranges are also possible (e.g., greater than or equal to 0.01 and less than or equal to 30).

In some embodiments, the capillary number of the outer fluid is greater than or equal to 0.001, greater than or equal to 0.01, greater than or equal to 0.1, greater than or equal to 1, greater than or equal to 10, or greater than or equal to 20. In certain embodiments, the capillary number of the outer fluid is less than or equal to 25, less than or equal to 10, less than or equal to 1, less than or equal to 0.1, or less than or equal to 0.01. Combinations of these ranges are also possible (e.g., 0.001-25).

In certain embodiments, the capillary number of the inner fluid is larger than the capillary number of the outer fluid. The capillary number of a fluid is expressed as:

$${C}_{a} = \frac{( μ * V )}{σ} \qquad ( Equation 11 )$$

where μ (mu) is the dynamic viscosity of the fluid, V is the average linear velocity of the fluid, and σ (sigma) is the interfacial tension between the inner and outer fluids.

In some embodiments, the orientation of the system (e.g., the needle and/or chamber) affects the timescale of eccentricity. For example, FIG. 8B demonstrates that, in accordance with certain embodiments, a T_{c}/tₑ of less than or equal to 1 is easier to achieve with the system (e.g., the needle and/or chamber) closer to vertical (90° from a line perpendicular to gravity), and more difficult to achieve closer to horizontal (0° from a line perpendicular to gravity).

In accordance with some embodiments, the longitudinal axis of the needle is within 45 degrees of a line perpendicular to gravity for at least one period of time. For example, in some cases, the longitudinal axis of the needle is within 30 degrees, 15 degrees, or 0 degrees of a line perpendicular to gravity for at least one period of time. In some embodiments, the period of time is between initiating flow of the inner fluid and/or outer fluid and ejection of the inner fluid and/or outer fluid from the needle. For example, in certain cases, the period of time is at least a portion of time (e.g., at least 50%, at least 75%, at least 90%, or the entirety of the time) between the initiating flow and the ejection from the needle.

As discussed above, in some embodiments, it is beneficial for lower amounts of the outer fluid to be ejected compared to the amount of inner fluid ejected (e.g., such that a patient is not exposed to large amounts of a lubricating fluid). In certain embodiments, the volumetric flow rate of the inner fluid is greater than the volumetric flow rate of the outer fluid. According to some embodiments, the volumetric flow rate of the inner fluid is ≥10⁻²×γπdₙ²/μᵢ. For example, in certain cases, the volumetric flow rate of the inner fluid is ≥5×10⁻²×γπdₙ²/μᵢ or ≥10⁻¹×γπdₙ²/μᵢ. In accordance with certain embodiments, the volumetric flow rate of the outer fluid is ≥10⁻³×γπdₙ²/μ_{O}. For example, in some instances, the volumetric flow rate of the outer fluid is ≥10⁻³×γπdₙ²/μ_{O}. For the volumetric flow rate, dₙ is the diameter of the needle, γ (gamma) is the surface tension of the two fluids, and μ is the dynamic viscosity of the fluid (where the i denotes the inner fluid and the o denotes the outer fluid).

In some embodiments, the concentration of a solubilized or suspended species (e.g., a drug) in the inner fluid can be significantly larger than in an identical article, system, and/or method without the outer fluid axially surrounding the inner fluid. For example, in some cases, the ratio of the concentration of a solubilized or suspended species (e.g., a drug) in the inner fluid according to certain embodiments disclosed herein compared to an identical article, system, and/or method without the outer fluid axially surrounding the inner fluid is greater than or equal to 1.1:1, greater than or equal to 1.5:1, greater than or equal to 2:1, greater than or equal to 5:1, greater than or equal to 10:1, greater than or equal to 50:1, greater than or equal to 100:1, or greater than or equal to 250:1. In some embodiments, the ratio of the concentration of a solubilized or suspended species (e.g., a drug) in the inner fluid according to certain embodiments disclosed herein compared to an identical article, system, and/or method without the outer fluid axially surrounding the inner fluid is less than or equal to 500:1, less than or equal to 250:1, less than or equal to 100:1, less than or equal to 50:1, less than or equal to 10:1, less than or equal to 5:1, or less than or equal to 2:1. Combinations of these ranges are also possible (e.g., 1.1:1 to 500:1).

In some embodiments, the articles, systems, and/or methods disclosed herein have a reduced pressure during injection compared to an identical article, system, and/or method without the outer fluid axially surrounding the inner fluid. For example, in some cases, the ratio of the pressure during injection compared to that of an identical article, system, and/or method without the outer fluid axially surrounding the inner fluid is less than or equal to 0.9:1, less than or equal to 0.7:1, less than or equal to 0.5:1, less than or equal to 0.3:1, less than or equal to 0.1:1, or less than or equal to 0.01:1. In some embodiments, the ratio of the pressure of the during injection compared to an identical article, system, and/or method without the outer fluid axially surrounding the inner fluid is greater than or equal to 0.001:1, greater than or equal to 0.01:1, or greater than or equal to 0.1:1. Combinations of these ranges are also possible (e.g., 0.001:1 to 0.9:1 or

Certain of the embodiments disclosed herein can provide one or more of several benefits, including reduced contamination, reduced needle clogging, reduced protein inactivation (e.g., when the inner fluid comprises a protein), increased concentrations of formulations (e.g., the inner fluid may be a high concentration drug formulation), increased viscosity of fluids, increased feasibility of subcutaneous administration (rather than intravenous administration), smaller needles, shorter injection times, reduced pain, fewer doses, reduced hydrodynamic resistance in the needle, reduced shear forces on the inner fluid, and/or reduced pressures. Examples of benefits that may arise from subcutaneous administration (which frequently require higher concentrations) rather than intravenous administration, in some embodiments, include increased feasibility of self-administration, reduced hospitalization, reduced treatment costs, and/or increased patient compliance.

In some embodiments, the systems described herein can inject viscous fluids without the use of larger needle gauges or prolonged injection times, which can cause pain. Moreover, in certain embodiments, the systems described herein can inject high concentration formulations without the use of syringe pumps, which can cause pain and can require a hospital setting. Additionally, in accordance with some embodiments, the systems described herein can inject viscous fluids without the use of needle free jet injectors, which frequently result in contamination and high costs. Further, in accordance with certain embodiments, the systems described herein can inject viscous fluids without particle encapsulation, which frequently results in protein inactivation, density based separation, needle clogging, and a higher degree of manufacturing complexity. The lack of a practical methodology to inject high viscosity formulations has not only limited the applicability of subcutaneous biologic formulations, but also hinders the development of new formulations as developers are forced to design formulations with lower viscosities. Therefore, there remains a pressing need to achieve injectability through a simple and inexpensive injection technique with minimal additions to the pharmaceutical manufacturing process and without risk of cross contamination.

The following examples are intended to illustrate certain embodiments of the present invention, but do not exemplify the full scope of the invention.

### EXAMPLES

Highly concentrated biologic drug formulations that can be delivered via subcutaneous injections offer tremendous benefits to global health, but they generally cannot be injected via commercial syringes and needles due to their high viscosities. Current approaches to solve this problem face several challenges ranging from cross contamination and high cost to needle clogging and protein inactivation. Discussed herein, is a simple method to enhance injectability using an axially lubricated flow, where the transport of highly viscous drugs through a needle was made easier, thanks to co-axial lubrication by a less viscous fluid. A phase diagram was established to obtain axially lubricated flow while minimizing the volume fraction of lubricant. This technique resulted in up to a 7× reduction in injection pressure for the largest viscosity ratio that was tested. Finally, these findings were implemented into the design and fabrication of a double barreled syringe that significantly expands the range of injectable concentrations of several biologic formulations.

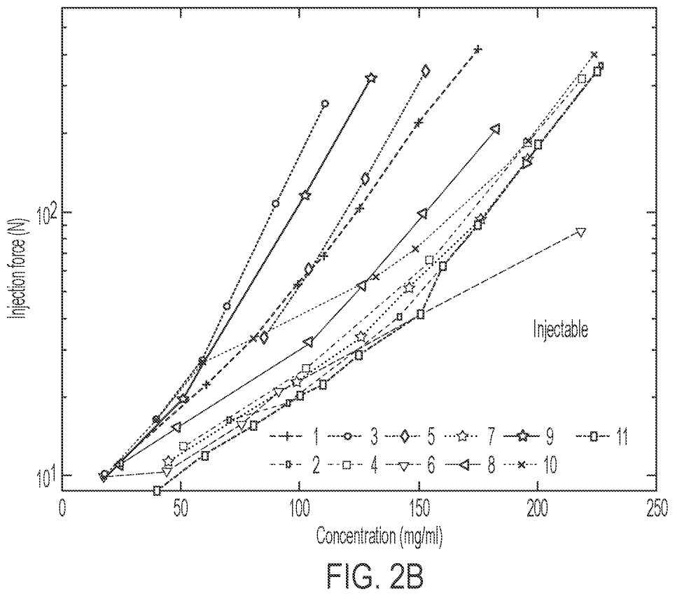

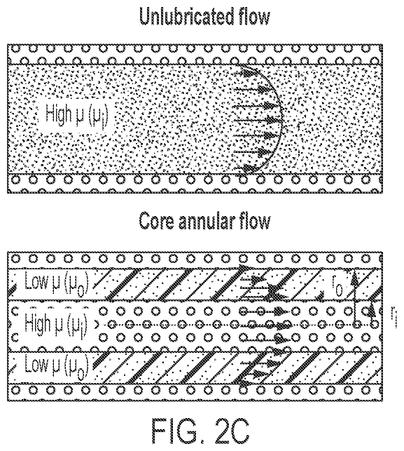

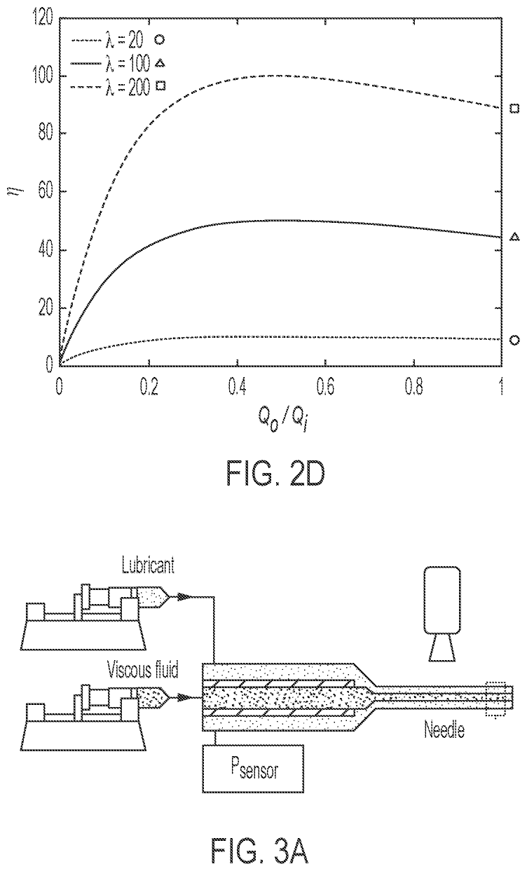

Current biologics are predominantly administered in low concentrations (<30 mg/ml) via intravenous injections and doses range from 5-700 milligrams. Over the past few years however, subcutaneous injection has emerged as an alternative delivery route as it (i) enables self-administration, (ii) reduces hospitalization and treatment costs, and (iii) increases patient compliance. Unlike intravenous injections, subcutaneous injections generally require formulations that are far more concentrated (>100 mg/ml) as injection volumes are limited to 1-1.5 ml per dose. This constraint is due to high back pressures that can develop in subcutaneous tissue at larger volumes. A non-linear relationship between formulation concentration and viscosity makes subcutaneous formulations very viscous and therefore harder to inject, as illustrated on FIG. 2A: a highly viscous fluid (top) spreads significantly less than a low viscosity fluid (bottom), when injected in a sponge at the maximal force that could be applied manually (around 50N). Consequently, the applicable force sets a limit to the concentrations of current formulations (FIG. 2B). FIG. 2B shows the injection force (for a flow rate of 4 ml/min through a 27 G needle) as a function of concentration for eleven monoclonal antibody solutions of the IgG1 isotype. This figure highlights the fact that a large range of formulation concentrations require more than 50N to be injected—the average maximum force that can be applied in a pinching motion.

### Example 1

Discussed herein is a technique to enhance the injectability of highly concentrated drug formulations using axially lubricated flows. In this technique, a low viscosity fluid axially lubricated the transport of immiscible viscous drugs through the needle (FIG. 2C). This not only reduced the hydrodynamic resistance in the needle but also reduced shear forces on the payload material (inner fluid).

The goal of this technology was to develop a device that uses axially lubricated flows to inject viscous formulations more easily. To realize this, the flow regimes observable in this device were reported and a regime map was established to indicate the flow rates and viscosity ratios at which axially lubricated flow was achievable in a needle. Finally, a co-axial, double barreled syringe was designed, fabricated, and tested to exhibit the capability of this technique to inject high concentration drugs.

### Results

The setup shown in FIG. 3A was used to study the dynamics of axially lubricated flows through a needle. Two syringe pumps were used to drive the inner viscous fluid and the outer lubricating fluid through a fluidic cross to establish the axially lubricated flow. A digital pressure sensor at the cross measured the pressure drop through the needle. A transparent needle was used to visualize the flow, and the dimensions of all components were chosen so that their hydrodynamic resistances are negligible compared to that of the needle.

Due to the volume and dosing constraints of subcutaneous biologic injections mentioned earlier, volume fractions (of viscous payload) lower than 55% were not considered. Therefore, the flow rate of the viscous fluid was fixed at 1 ml/min and the flow rate of the lubricant was varied from 0.1 to 0.8 ml/min. These flow rates were chosen as they were in the range of flow rates that would be required for a practical injection. FIG. 3B shows a map of the observed flow regimes for different flow rate and viscosity ratios. Two regimes occurred in the phase space: a viscous displacement regime at low outer fluid flow rates, and an axially lubricated flow regime as the lubricant flow rate increased. In the viscous displacement regime, the viscous fluid first filled the entire cross-section of the needle and forced both fluids to back-flow into the lubricant inlet. However, this backflow could not be sustained due to the constant mass flux that was imposed on the lubricant, resulting in a sudden overflow of the lubricant into the needle. This flow decreased until being completely hindered once again, and the process repeated. This cyclic behavior is shown in the temporal diagram of a cross section of the needle (FIG. 3C) and it led to unsteady and significantly worse lubrication compared to the axially lubricated flow regime (FIG. 3D).

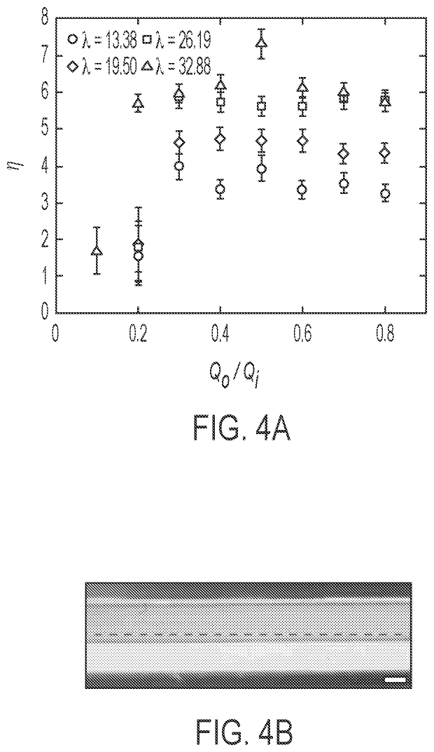

FIG. 4A reports the experimental pressure reduction coefficient (mean±std. error) as a function of the ratio between the lubricant flow rate and the viscous fluid flow rate, for different viscosity ratios. Experiments corresponding to the viscous displacement regime (Qₒ/Qᵢ≤0.2) exhibited larger errors due to the cyclic nature of the regime. Thus, the average pressure reduction factors in this regime were much lower than in the case of the axially lubricated flow regime (Qₒ/Qᵢ>0.2).

A system with eccentricity due to buoyancy was studied, as shown in FIG. 4B, which is a digital photograph of a side view of the needle. The experimental measurements for the pressure reduction coefficient are shown in FIG. 4A. While pressure reductions were still observed, a significant difference in the magnitude of the pressure reduction coefficients was observed compared to a concentric system. This lower performance originated from the eccentricity caused by the density difference between the two phases.

### Discussion

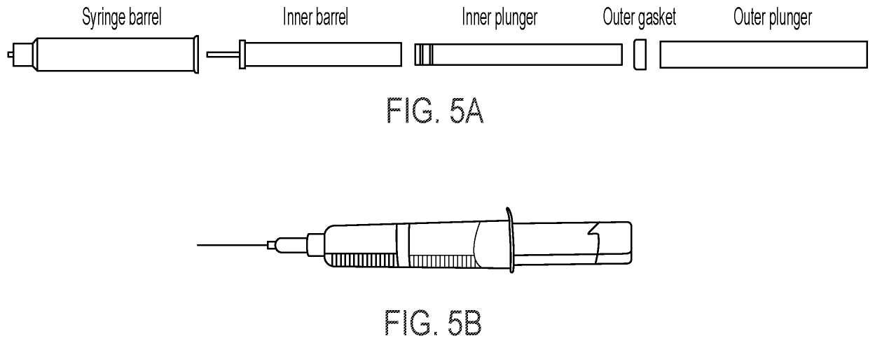

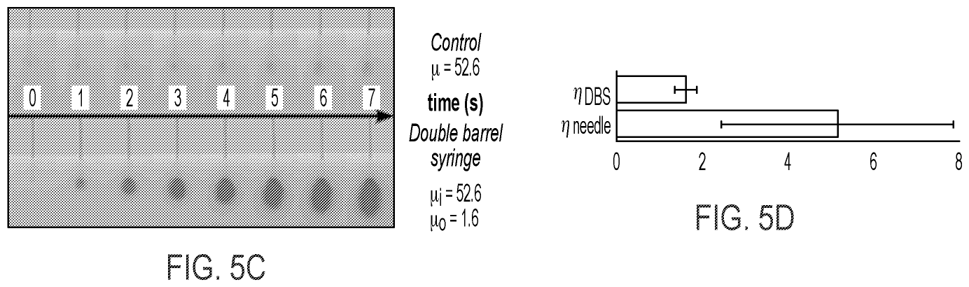

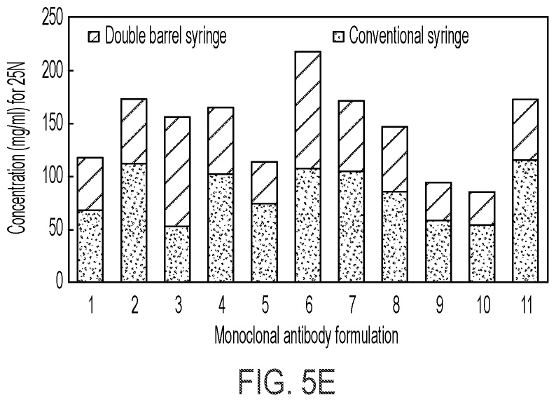

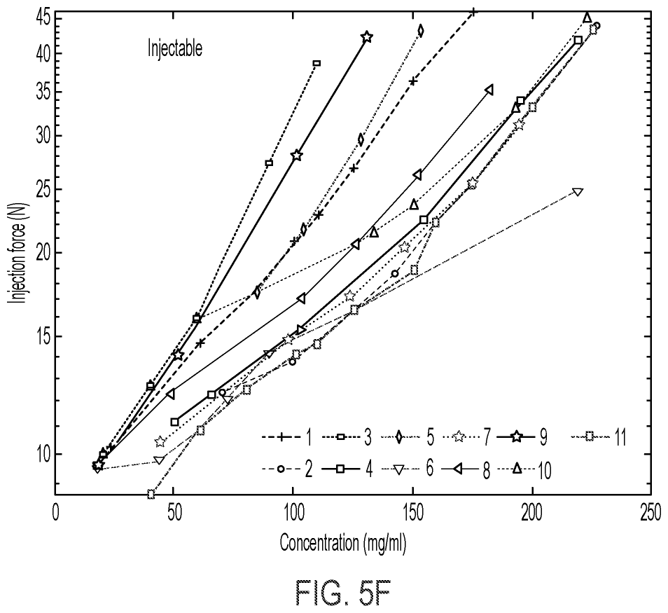

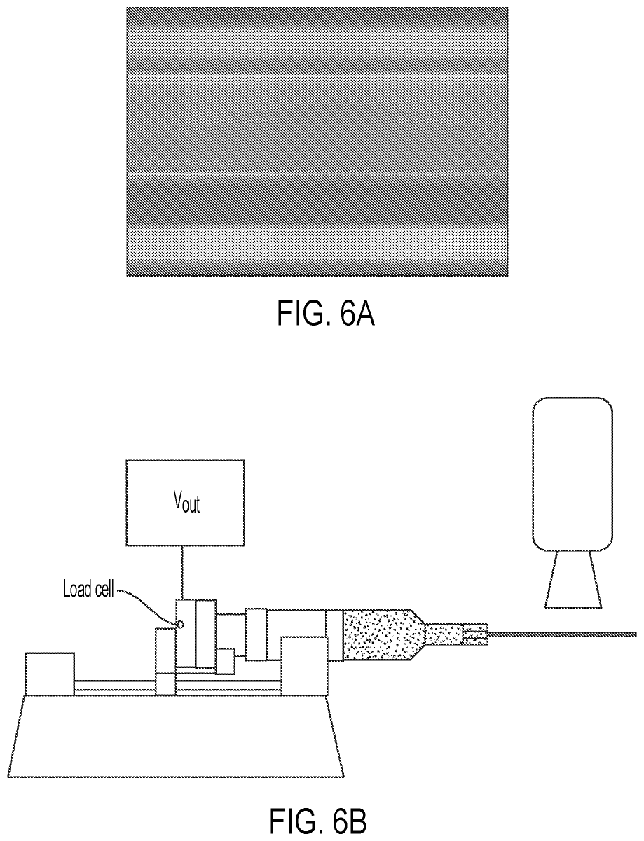

To implement this knowledge into a practical device, the double barreled syringe, shown in FIGS. 5A-5B, was designed and fabricated. The double barreled syringe included an outer barrel that contained the lubricant and an inner barrel that held the viscous payload. A six milliliter syringe barrel was used as the outer barrel. The fluids were driven by corresponding outer and inner plungers with a movable outer gasket that facilitated leak-proof operation. The dimensions of the barrels were chosen so that during the displacement of the plungers, the ratio of lubricant flow rate to viscous fluid flow rate was approximately 0.59: which is significantly above the threshold value of 0.2 that was observed to be needed to sustain an axially lubricated flow. The simplicity of this design made it easy to manufacture, as it could be made using injection molding or blow-fill seal processes, facilitating similar ease-of-use and cost as current commercial medical syringes and needles. FIG. 6A shows the axially lubricated flow established in a needle connected to the double barreled syringe, indicating that it indeed operated in the axially lubricated flow regime.

Visual evidence of the enhancement in manual injectability is shown in FIG. 5C, where better liquid spreading was shown in a sponge when a high viscosity fluid was injected using the double barreled syringe (top) compared to a commercial syringe (bottom). In order to quantify this improvement, the forces needed to inject a water-glycerin solution (26.3 cP) with the double-barreled syringe and a commercial syringe for the same volume flow rate was compared. The injection force was quantified with a load cell mounted on a syringe pump (FIG. 6B). The measured forces were used to calculate the force reduction coefficients, η_{DBS} and η_{needle} which were defined as follows:

$${η}_{D B S} = \frac{{F}_{c ommercial syringe}}{{F}_{Double barreled syringe}} \qquad ( Equation 12 )$$

$${η}_{needle} = \frac{{F}_{c ommercial syringe} - {F}_{commercial syringe - without needle}}{{F}_{Double barreled syringe} - {F}_{Double barreled syringe - w i thout needle}} \qquad ( Equation 13 )$$

FIG. 5D shows the experimental force reduction coefficients obtained from this double barreled syringe. Both the comparator and double barreled syringe were run with and without the needle in order to quantify the resistance of the barrels. This proof-of-concept design suffered from significant friction between the barrels and the plungers, resulting in a low value for 17DBS. However, this friction can be largely eliminated by using existing syringe manufacturing techniques (such as injection molding to make more appropriately sized gaskets). The larger variability observed for the needle was due to the error propagation operations carried out to isolate the resistance of the needle alone. When the contributions of the barrels were removed, a force reduction coefficient of 5 in the needle was observed.

Such a significant force reduction coefficient demonstrated the promise of the technique to increase the threshold concentrations of biologic drugs. To further emphasize this, FIG. 5E shows the increases in concentrations that were possible for eleven monoclonal antibody solutions reported in literature while keeping injection force at a nominal 25N. It revealed that it was possible to double (formulation 6) and even triple (formulation 3) the injectable concentration for certain monoclonal antibody formulations using this double barreled syringe. Finally, when considering the maximum force that can be applied in a pinching motion (50N), FIG. 5F demonstrates that the regime of manually injectable formulations can be significantly expanded by using the double barreled syringe. Furthermore, the reduction of force for lower concentration formulations could facilitate faster injections or the use of smaller needles, resulting in less pain for patients.

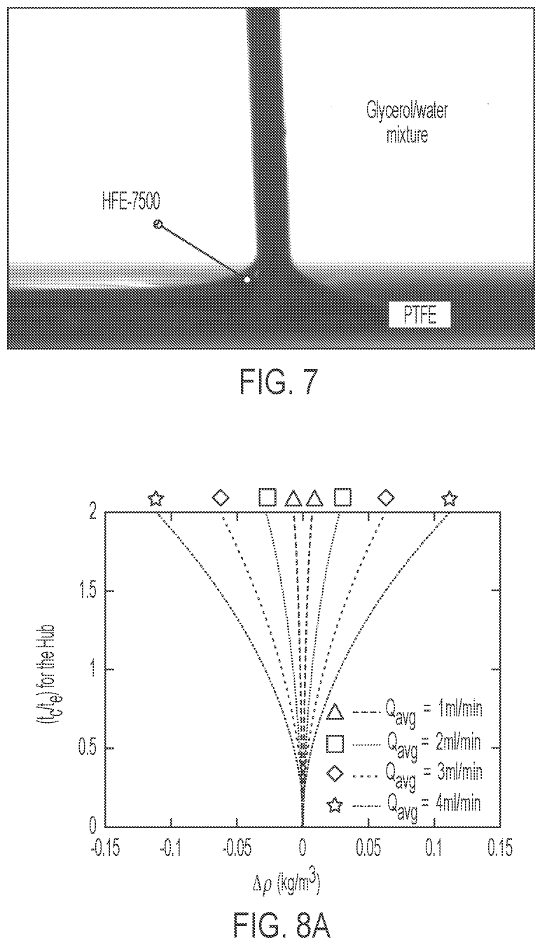

It was discovered that the relative wettability of the inner and outer fluids with respect to the interior surface of the chamber and/or needle were important. Indeed, if the inner fluid preferentially wets the interior needle surface, the outer lubricant flow might fail, leading to an unstable axially lubricated flow and therefore, tremendous loss in pressure reduction. This, combined with its biocompatibility, was why HFE-7500 was chosen as the lubricant for further experiments, as HFE 7500 was more wetting towards the needle than the viscous aqueous payload. This is shown in FIG. 7 where the HFE 7500 was observed to be fully wetting on PTFE in the presence of glycerol (the model viscous payload). Without wishing to be bound by any particular theory or mode of operation, it is believed that this favorable wetting may have been the reason that the flow was not completely eccentric in these experiments (E=0.98). Other biocompatible oils, such as squalene, which have been shown to promote adjuvancy effects resulting in a faster immune response to injections, could also be used as the lubricants.

The benefits observed in the axially lubricated flow based injection technique could be expanded to other subcutaneous delivery methods as well. Micro-needle patches for example, could be made with smaller needles or could be used for shorter periods of time, if the resistance to flow is reduced using axially lubricated flows. This methodology also holds substantial promise for applications beyond biopharmaceuticals. For example, the lubricating effect of axially lubricated flows could be expanded to other high viscosity or non-Newtonian fluids that need to be injected, such as bone putty or hydrogels. The reduced shear in such flows could also be applied to handle and dispense sensitive or primary cells where low shear is essential to prevent damage.

Demonstrated here, was a simple yet efficient technique to enhance the injectability of high concentration biopharmaceuticals using axially lubricated flows. A regime map of flow rates and viscosity ratios required to attain stable axially lubricated flow while minimizing the flow rate of the lubricant were established. Significant pressure reduction was achieved in axially lubricated flows for a variety of payload viscosities. Experimentally, up to a 7× reduction in pressure for λ≈33 was achieved. In addition, the role of buoyancy-based eccentricity was examined. Finally, the knowledge was applied to design, fabricate and test a prototype double barreled syringe. A substantial pressure reduction was shown, up to 5× reduction fork λ=26, in this syringe, therefore significantly expanding the regime of injectable viscosities for biologics without increasing costs, risk of cross contamination or manufacturing complexity.

### Methods

### Fluid Preparation and Characterization

### Fluid Preparation

Mixtures of glycerol and water of different viscosities were used as the inner fluid in all experiments. HFE-7500+2 wt % fluorosurfactant (obtained from RAN Biotechnologies) was used as the lubricant (outer fluid).

### Rheology

A TI ARG-2 rheometer was used to measure the viscosity of all the samples. A 40 mm 2° cone geometry was used to measure the viscosity of all the glycerol solutions. Stepped flow tests were done where the shear rate was varied from 10 s⁻¹ to 500 s⁻¹. A 60 mm plate geometry was used to measure the viscosity of HFE 7500. Here, the shear rate was varied from 1 s⁻¹ to 100 s⁻¹.

### Interfacial Tension

Interfacial tension measurements were done using a Rame′-Hart contact angle goniometer. The pendant drop method was used to measure the interfacial tension where a glycerol drop was suspended in a bath of HFE 7500+2 wt % fluorosurfactant.

### Experimental Setups

### Pressure Reduction Measurements

Harvard apparatus PHD ULTRA™ syringe pumps were used to drive the fluids. A fluidic cross with ⅛″ NPT female fittings was used to establish the axially lubricated flow. Specifically, the high viscosity fluid flowed through a 1/16″ OD tube that traveled through the entire cross and a luer adapter before entering the hub of the needle. The lubricant was brought through one of the branches in the cross and was allowed to exit to the needle coaxially with the inner viscous fluid. A 304.8 μm ID, 2″ long PTFE needle was used in all experiments. The final branch of the cross housed a Honeywell® 26PC series pressure sensor. The sensor is connected to a DC power supply and its output is measured using a Keithley® 2450 sourcemeter operating as a voltmeter.

### Testing the Double Barreled Syringe

An Omega engineering LC 307 series load cell was used to measure the force on the plunger. The load cell was attached to the driving plate of the syringe pump and a 3D printed adapter was used, such that the plungers made contact only with the load cell during operation.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article for delivery of a fluid, comprising:
a chamber;
a needle fluidically connected to the chamber;
an inner fluid extending from the chamber into the needle and flowing through the needle; and
an outer fluid extending from the chamber into the needle, axially surrounding the inner fluid, and flowing through the needle;
wherein the outer fluid mixes with the inner fluid at most 50% while in the needle.

2. An article for delivery of a fluid, comprising:
a chamber; and
a needle fluidically connected to the chamber;
wherein the article is configured such that, when an inner fluid and an outer fluid are transported through the needle, the outer fluid axially surrounds the inner fluid, and the outer fluid mixes with the inner fluid at most 50% while in the needle.

3. (canceled)

4. The article of claim 1, wherein the outer fluid and/or the inner fluid is soluble in the other in an amount of more than 0.001 mass fraction.

5. An article for delivery of a fluid, comprising:
a chamber;
a needle fluidically connected to the chamber;
an inner fluid extending from the chamber into the needle and flowing through the needle; and
an outer fluid extending from the chamber into the needle, axially surrounding the inner fluid, and flowing through the needle;
wherein the article has an eccentricity parameter (E) of less than 1 when a longitudinal axis of the needle is within 45 degrees of a line perpendicular to gravity for at least one period of time.

6-9. (canceled)

10. The article of claim 1, wherein the outer fluid is a Newtonian fluid.

11. The article of claim 1, wherein the outer fluid is a yield stress fluid.

12. The article of claim 1, wherein a ratio of the viscosity of the inner fluid to the viscosity of the outer fluid (μᵢ/μₒ) is greater than 1.

13. The article of claim 12, wherein the ratio of the viscosity of the inner fluid to the viscosity of the outer fluid (μᵢ/μₒ) is greater than 10.

14. The article of claim 1, wherein, for the inner fluid, the outer fluid, and an interior surface of the needle, Sₒₙ₍ᵢ₎≥0.

15. The article of claim 1, wherein a capillary number of the inner fluid is greater than or equal to 0.01 and a capillary number of the outer fluid is greater than or equal to 0.001.

16. The article of claim 15, wherein the capillary number of the inner fluid is larger than the capillary number of the outer fluid.

17. The article of claim 1, wherein an interior surface of the needle comprises a texture.

18. (canceled)

19. The article of claim 1, wherein an interior surface of the needle comprises a coating.

20. (canceled)

21. The article of claim 1, wherein an interior surface of the needle comprises a texture for which φₛ≤0.5.

22. (canceled)

23. The article of claim 1, wherein the outer fluid preferentially wets an interior surface of the chamber.

24-30. (canceled)

31. The article of claim 1, wherein the needle has a length of greater than or equal to 5 microns.

32. The article of claim 1, wherein the needle has a length of at least 10 mm.

33. The article of claim 1, wherein the article is a syringe needle system.

34. The article of claim 1, wherein the article is non-manually actuated.

35. A method of delivery of a fluid, comprising:
within the article of claim 1, initiating flow of at least a portion of the inner fluid and at least a portion of the outer fluid, such that at least a portion of the inner fluid and at least a portion of the outer fluid are transported from the chamber to the needle and ejected from the needle.

36-47. (canceled)
